(19) United States
(12) Patent Application Publication
(10) Pub. No.: US 2003/0044869 A1
(43) Pub. Date: Mar. 6, 2003

(54) Method and apparatus for detecting protein in a sample

(76) Inventors: Jupiter M. Yeung, Rockville, MD (US); Carmen Diaz Westphal, Baltimore, MD (US)

Correspondence Address: COVINGTON & BURLING, ATTN: PATENT DOCKETING, 1201 PENNSYLVANIA AVENUE, N.W., WASHINGTON, DC, 20004-2401, US

(21) Appl. No.: 09/944,153

(22) Filed: Sep. 4, 2001

(51) Int. Cl. G01N 33/53; G01N 33/567; G01N 33/537; G01N 33/543

(52) U.S. Cl. 435/7.92

(57) ABSTRACT

Apparatus and method for detecting a protein in a sample, such as a sample flushed through a food processing system. If the sample or food processing system contains the protein in detectable amounts, a test membrane will change color. The invention thus provides a simple semi-quantitative test for the presence of a protein.

### BACKGROUND OF THE INVENTION

1. Field of the Invention

The present invention relates to the detection of trace amounts of proteins, such as allergens, in food. More particularly, the present invention relates to a method and apparatus for detecting a protein in a sample and for detecting the inadvertent presence of a protein in a food processing system.

2. Related Art

Food allergy is a problem in our society. The true prevalence of immediate food hypersensitivity reactions in adult and pediatric populations is unknown, as is the actual mortality rate. The Mt. Sinai School of Medicine recently estimated that 150-200 people die each year due to food allergy. It is believed that approximately 2% of the population in the United States (approximately 6-7 million people) is affected by food allergy and that as high as 8% of children less than 3 years old are allergic to foods. In fact, the number of allergic food reactions appears to be increasing, particularly in developing countries over the last decade. In approximately one-third of U.S. households, people have altered their dietary profiles because someone in the household believes particular foods cause allergic reactions. Food allergy reactions are much more common in the pediatric population than the adult population. The causes are not fully understood. The increased prevalence of food allergy early in life may be due to increased mucous membrane permeability and an immature immune system. Indeed, some food allergies are outgrown, in particular milk and egg allergy are most commonly outgrown. Additionally, there is clearly a genetic predisposition to hypersensitivity reactions. Any organ system can be involved in a food hypersensitivity reaction; however the most common organ systems involved are the skin, gastrointestinal tract, and respiratory system.

As reported in the Journal of AOAC International (Yeung, J. M. and Collins, P. G. (1996) J. AOAC International Vol. 79, No. 6), peanuts are one of the most common food allergies in both children and adults. A survey of severe allergic reactions to foods over a two year period in 73 Colorado emergency departments by S. A. Bock ((1992) J. Allergy Clin. Immunol. 90, 683-658), showed that 28% of the affected patients may have reacted to peanuts. Another study reports that 21% of patients with a food-associated allergy were allergic to peanuts. May, C. D. & Bock, S. A. (1978) Allergy 33, 166-171. And although fatal and near fatal anaphylactic reactions to foods are rarely reported, the Sampson et al. article (“Fatal and near-fatal anaphylactic reaction to food in children and adolescents,” N. Engl. J Med. 1992; 327:380-4) noted 13 cases involving accidental exposure. Four of these 13 accidents were caused by peanuts.

Although food allergy remains relatively uncommon, it is an established definite clinical entity. Clinical acceptance of food sensitivity was delayed by the lack of laboratory tests, in spite of clear clinical history described by patients suffering from food allergy. The gold standard for documenting whether an individual has an adverse reaction to certain foods is a double-blind, placebo-controlled food challenge. Diagnosis is based on the findings of a thorough history, physical examination and diagnostic testing to eliminate other diagnoses.

Once food allergy is diagnosed, strict dietary avoidance is the principle and only effective method of management. Education about emergency management is also extremely important. Most do not consider immunotherapy effective for the treatment of food allergy at this time. Because even trace levels of peanuts, for example, can elicit an adverse to fatal reaction, unintentional exposure to the offending food allergen may have devastating consequences to sensitive individuals. Given this sensitivity and the enormous changes in demographics, consumer life-styles, food technologies and public health policies in this country over the past several decades, undeclared food allergens are a tremendous challenge to food labeling policies. In addition to potentially inadequate food labeling, cross-contact in food processing facilities may pose serious threats to sensitive individuals. However, detecting trace levels of unintentional common food allergens such as peanuts, in our food supply has been very difficult. The food industry is in need of accurate, rapid and economical methods to detect the inadvertent presence of food allergens in order to ensure food safety, to prevent potentially fatal hypersensitivity reactions, and to avoid costly recalls.

Food contaminant testing is currently dominated by chromatographic methods such as Gas Chromatography (GC) and High Performance Liquid Chromatography (HPLC). However, immunoassays such as immunoblot or enzyme-linked immunosorbent assay (“ELISA”) are the current method of choice for protein analysis. Immunoassays differ from chromatographic methods of analysis in that the technology advancement is in the antibodies developed rather than in the instruments. The prevalent 96-well microtiter plate format allows large numbers of samples to be analyzed simultaneously and data reduction can be automated. Despite the advancements made by the current ELISA test methods, there remains a need in the art for a portable method and apparatus for the detection of food allergens. There is a particular need for an accurate, qualitative, fast, easy and economical method to detect the presence of food allergens. The present invention, the description of which is fully set forth below, solves the need in the art for such improved methods and apparatus.

### SUMMARY OF THE INVENTION

One aspect of the present invention relates to a method and apparatus for detecting the presence of a protein in a sample. The sample can consist of, for example, a liquid wash from a food processing system or an extract from an end food product. The apparatus consists of an insoluble membrane that is placed onto a test strip. Part of the insoluble membrane, the test spot, is coated with an antibody (the captive antibody) against whatever protein is to be detected. An exemplary captive antibody is an anti-peanut antibody. The anti-peanut antibody may be obtained from the serum of a rabbit that has been inoculated with peanut protein. There is also a control spot on the membrane that has been coated with an anti-peroxidase enzyme antibody (the control antibody), a monoclonal antibody clone P6-38, purchased from Sigma (catalog number P2419).

The invention is used by dipping or immersing the test strip into the sample, and then rinsing the insoluble membrane. If the corresponding protein is present in the sample at a detectable level (ranging from about 0.5 to about 10 ppm and above), then the corresponding protein from the sample links to the captive antibody on the test strip. Then the test strip is dipped into a signal antibody solution consisting of an antibody conjugated to a peroxidase enzyme (the signal antibody or the signal antibody conjugate). If the corresponding protein is present in the sample and has linked to the captive antibody, then the corresponding protein from the sample will also link to the signal antibody thereby forming an antibody-antigen-antibody complex. After dipping the test strip in the signal antibody solution, the test strip is rinsed.

After rinsing the test strip, it is dipped or immersed in a substrate solution. The substrate solution is preferably colorless, and contains a substrate that chemically reacts with the peroxidase enzyme. This chemical reaction causes the control spot and the test spot to change color. The test spot changes color after the protein from the sample links to the anti-protein antibody coating the test spot and then to the signal antibody from the peroxidase enzyme antibody conjugate solution. The control spot changes color after the signal antibody conjugate links to the control anti-peroxidase enzyme antibody coated on the control spot.

After the test strip has dried, it can be inspected to identify any difference in color between the color of the test spot and the color of the control spot. Test results are interpreted as follows: a test spot that shows the same or more color intensity than the control spot means that the sample is positive (it contains the protein), whereas a test spot that shows either less color than the control spot or no color at all, means that the sample is negative (and does not contain the protein in levels that can be detected by the present invention), and if the control spot does not change color or intensity after the test has been completed, then the test is invalid.

The test strip can also further contain a second captive or a plurality of different captive antibodies, wherein the second captive antibody and/or the plurality of captive antibodies are different from each other, the signal antibody, and the control antibody. These captive antibodies are selected from the group of antibodies consisting of peanut, soy, milk, egg, fish, shellfish, tree nut and seeds. The test strip can also further contain a second signal antibody conjugate or a plurality of different antibodies conjugated to a peroxidase enzyme, wherein the second antibody conjugate or the plurality of signal antibodies conjugated to a peroxidase enzyme are different from the captive antibody, control antibody, and signal antibody conjugate. The antibodies conjugated to the peroxidase enzyme are selected from the group of antibodies consisting of peanut, soy, milk, egg, fish, shellfish, tree nut and seeds.

Features and Advantages

It is a feature of the present invention that the method of detection is direct, in contrast to the reported allergen ELISAs which use the 96-well format. The method of detection is “direct” because an antibody is labeled on an affinity membrane to capture the antigen. Therefore, the resulting color is directly proportional to concentration of antigen or analyte. In other words, the resulting color will be stronger or brighter, the more antigen that is present in the sample, except that if the amount of antigen or analyte present in the sample is above a concentration of about 5 ppm, the intensity of the color will not increase due to saturation. Furthermore, if the amount of antigen or analyte present in the sample is below a concentration of about 0.5 ppm, there will be no detectable color change. In contrast, the conventional competitive 96-well ELISA method is an indirect method. It is an indirect or competitive method because the antigen (not the antibody) is labeled on the support. The antibody competes for binding of antigen present in the sample and antigen present on the support. Therefore, color is inversely proportional to concentration of antigen or analyte. It is also a feature of the present invention that the antibodies are created differently from those used in the reported ELISA tests. The antibodies used in the present invention, are obtained by inoculating rabbits with an immunogen (for example peanut proteins plus adjuvant) and then isolating the anti-peanut antibodies from their serum. The rabbits are immunized with different dosages of immunogen. Half the dose of primary dose, and one quarter of the booster doses are used for the immunization to enhance sensitivity and specificity. The antibodies used in other allergen ELISA tests are not obtained from animals immunized with different doses of immunogen and thus are not as sensitive or specific as those in the present invention.

It is a significant advantage of the present invention that it can detect trace amounts of proteins or allergens in food or in a food processing system faster and easier than previous methods of detection. A simple qualitative presence/absence test at defined threshold levels, such as the one contemplated by the present invention, should be the first step in testing food and/or food processing systems. Previous ELISA methods of testing are quantitative and take several hours to run and interpret. By reducing the amount of incubation, changing the test format, and the amount of solutions and reagents that must be added, the present invention can be run in less than 20 minutes. This makes the present invention extremely useful in food processing plants where it can be quickly determined whether a production line or equipment has been adequately cleaned prior to shifting to another production. This advancement can save the several hours to days of down-time on a food production or processing line to wait for results if using the prior methods of testing. Eliminating several hours to days of down-time increases efficiency and the amount of food that can be processed, which of course, ultimately saves money and increases net revenue. The food industry needs an accurate, rapid, portable and economical method such as the present invention, to detect the presence of food allergens in order to avoid unacceptable health risks to susceptible consumers and to avoid expensive and time consuming recalls.

The present invention provides a method and apparatus that are particularly advantageous for testing in the field. Water, buffer solution and a dropper are all that is needed to use the present invention. Conventional testing (previous ELISA tests) for proteins or allergens in food is more labor intensive and requires sophisticated laboratory equipment, instrumentation and skilled labor to perform the tests and to interpret the results. Conventional testing is cumbersome and is not suitable for use in the field. In contrast, the present invention is simple, entirely portable and user-friendly. It can be conducted in the field and the results can be immediately interpreted by one person with no technical training. The only required skill is the ability to read the instructions. This makes the present invention particularly useful and cost effective for commercial applications.

The present invention also advantageously creates a permanent record of the test results. Existing ELISA tests use soluble substrates that provide an indirect result. Current ELISA test results are indicated by color changes that are not permanent and that require an ELISA reader to convert color absorption units for quantification and to provide documentation. The substrate of the present invention is insoluble and thus provides a permanent record that can be preserved. The color change that results from testing a sample with the present invention is stable over time. If exposed to light for a length of time the background may darken. However, the contrast between the background and the test areas will remain clearly discernible. The ability to document the results of testing is an added advantage provided by the present invention. Documentation is particularly important, for example, to support good manufacturing practice (GMP) for the Food and Drug Administration (FDA). In such instances, permanent documentation that can be verified by others is particularly valuable.

Another advantage of the present invention is that the components are easier to store, have a longer shelf-life and are more durable than conventional methods of protein detection. The present invention provides an apparatus that can be stored in the refrigerator or on a shelf. ELISA test apparatus that consists of plastic microtiter wells coated with antibodies or antigen and the various solutions that are added to the wells, require refrigeration or freezing. In contrast, the apparatus of the present invention is composed of an affinity insoluble membrane mounted on a support. In contrast to existing methods of testing, the present invention can be dropped or shaken without compromising the test results.

Yet another advantage of the present invention is that is more sensitive than existing methods of allergen/protein testing. The present invention can detect protein in a sample at levels as low as about 0.5 parts per million (ppm), which is approximately 5-10 fold more sensitive than the corresponding ELISA apparatus.

### BRIEF DESCRIPTION OF THE FIGURES

The present invention is described with reference to the accompanying drawings. In the drawings, like reference numbers indicate identical or functionally similar elements.

FIG. 1 shows an exemplary embodiment of one configuration of an apparatus of the present invention;

FIG. 2 illustrates results of a test using one embodiment of a method and apparatus of the present invention;

FIG. 3 illustrates how one embodiment of the method and apparatus are used to detect a protein in a sample or to detect a contaminant in a food processing system;

FIG. 4 shows an exemplary embodiment of an antibody-antigen-antibody complex at a test spot after a sample is bound to a captive antibody and to a signal antibody, as well as an exemplary embodiment of what occurs at a control spot after a control anti-peroxidase enzyme antibody binds to a signal antibody conjugate.

### DETAILED DESCRIPTION OF THE PREFERRED EMBODIMENTS

Overview

The present invention provides a method and apparatus for detecting a protein in a sample or for detecting a contaminant in a food processing system. One aspect of the present invention relates to a method for detecting one or more proteins in a sample. In a typical application of the present invention, the protein is inadvertently or unknowingly present in the sample or in a food processing system. Although the present invention will be described below with respect to detecting a protein, the invention is not limited to detection of proteins. For example, it should be understood by one skilled in the art that the present invention can be used to detect other components, including but not limited to, cross-contaminating proteins or cross-contact proteins in a sample such as a sample obtained from a food processing system. Such cross-contact includes proteins such as peanut, soy, milk, egg, fish, shellfish, tree nuts and seeds, as well as other proteins readily apparent to one skilled in the art.

In one aspect of the invention, a kit for detecting the presence of a protein in a sample is provided and consists of an insoluble membrane attached to a test strip. An area (the test spot) of the insoluble membrane is coated with an antibody (the captive antibody) against whatever protein is being tested for, for example, an anti-peanut antibody. The polyclonal anti-peanut antibody is obtained from the serum of a rabbit that has been inoculated with peanut protein in the manner described above with different doses of immunogen. It should be apparent to one skilled in the art, that other antibodies with different doses of immunogen are suitable for use with the present invention, such as soy, milk, egg, fish, shellfish, tree nuts and seeds. There is a control spot on the membrane that has been coated with a control anti-peroxidase enzyme antibody available commercially from Sigma (monoclonal antibody clone P6-38, catalog number P2419). The apparatus also includes a reagent consisting of a signal antibody solution consisting of a peroxidase enzyme conjugated to an antibody and a second reagent consisting of a substrate solution that is preferably colorless.

The apparatus is used by contacting the test strip with the sample and with the signal antibody solution reagent, and if the protein is present in the sample, the protein links to the captive antibody and then to the signal antibody. Additionally, the control anti-peroxidase enzyme antibody present on the control spot links to the signal antibody conjugate after the test strip is contacted with the signal antibody conjugate reagent.

The kit is used to detect the presence of a protein in a sample by dipping or immersing the test strip into the sample and then rinsing the test strip with water for approximately 20 to 30 seconds. If a protein is present in the sample in detectable levels, the protein links to the captive antibody. Then the test strip is dipped or immersed into a signal antibody solution so that if a protein is present in the sample it will link to the signal antibody (the protein should have already linked to the captive antibody). The test strip is then rinsed with water for approximately 20 to 30 seconds. After the test strip is rinsed, it is dipped or immersed into a substrate solution that is preferably colorless (a second reagent in the kit of the present invention). The test spot will change color after the protein from the sample that is now linked to the captive antibody and to the signal antibody is dipped into the colorless substrate solution. The control spot will also change color after the control anti-peroxidase enzyme antibody links to the signal antibody conjugate and the control spot is immersed in the substrate solution. The test strip is rinsed again with water for approximately 2 seconds and then allowed to air dry completely in a dark place.

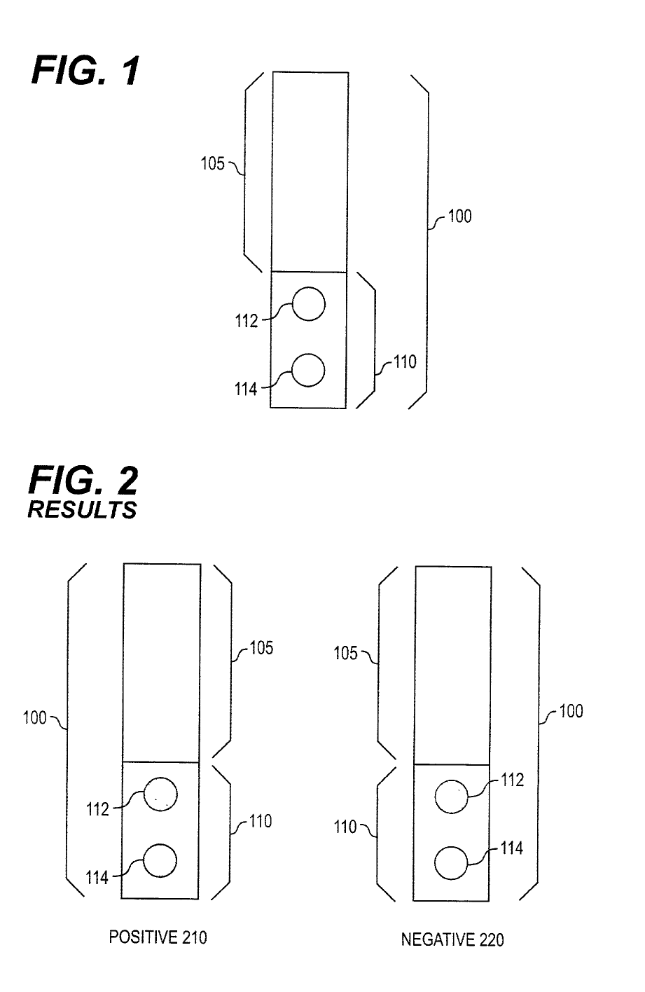

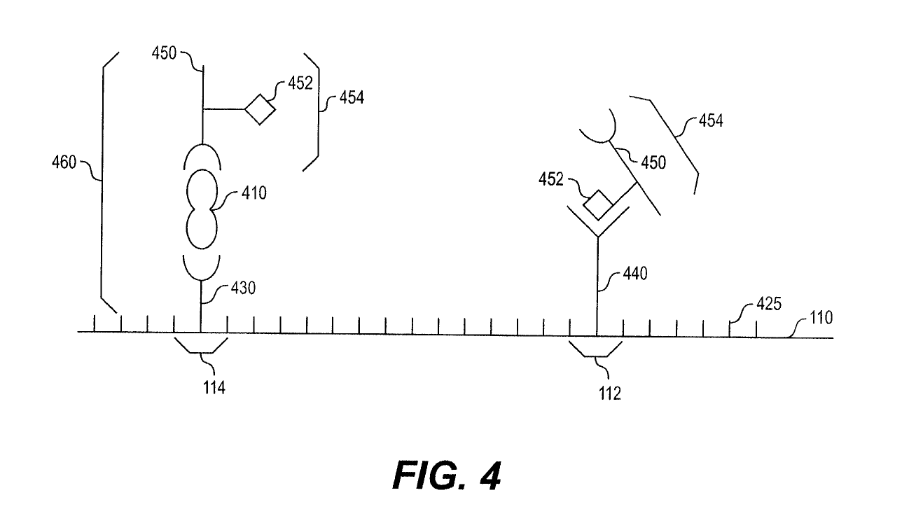

FIG. 1 illustrates one embodiment of the apparatus of the present invention. The apparatus comprises an insoluble membrane 110 composed of an affinity membrane called Optitran that is available commercially from Schleicher & Scheull (product number 68330; 0.45 μm pore size) and distributed in the U.S. by Sigma (Sigma #Z36,022-8). The membrane 110 is mounted to part of a test strip or support 100 made of Clear-Lay™ from Grafix Plastics which is composed of poly vinyl chloride (PVC) in 0.010″ thickness. Area 105 of support 100 is not covered by membrane 110. Area 105 can be held and/or labeled for example, by a user during conduction of the test. The apparatus is prepared as follows: the test spot 114 on the insoluble membrane 110 is coated with purified polyclonal anti-protein X captive antibodies obtained by inoculating rabbits with protein X and then isolating the anti-protein X antibodies from the rabbit serum using methods known to one skilled in the art, where protein X is the protein for which detection is desired. After coating test spot 114 with protein X captive antibodies and coating control spot 112 with anti-peroxidase enzyme antibody, insoluble membrane 110 is allowed to dry for approximately 30 minutes. Control spot 112 and test spot 114 will be explained in more detail below with respect to FIGS. 2 and 4. Insoluble membrane 110, including test spot 114 (which may also be referred to herein as “the portion”) and control spot 112 (which may also be referred to herein as “the quantity”), is then blocked with a blocking agent (425 as shown in FIG. 4) such as 1% poly-ethylene-glycol solution (“PEG”) in 5% dry milk. The blocking agent prevents the sample from binding to the insoluble membrane 110 in all areas of the membrane that are blocked. The membrane is rocked back and forth for approximately 30 minutes at room temperature. It is washed with phosphase buffered saline with Tween 20 (“PBST”) (0.1%) at a pH of 7.4, and rocked for two more minutes. The membrane 110 is washed three times using clean water for each rinse.

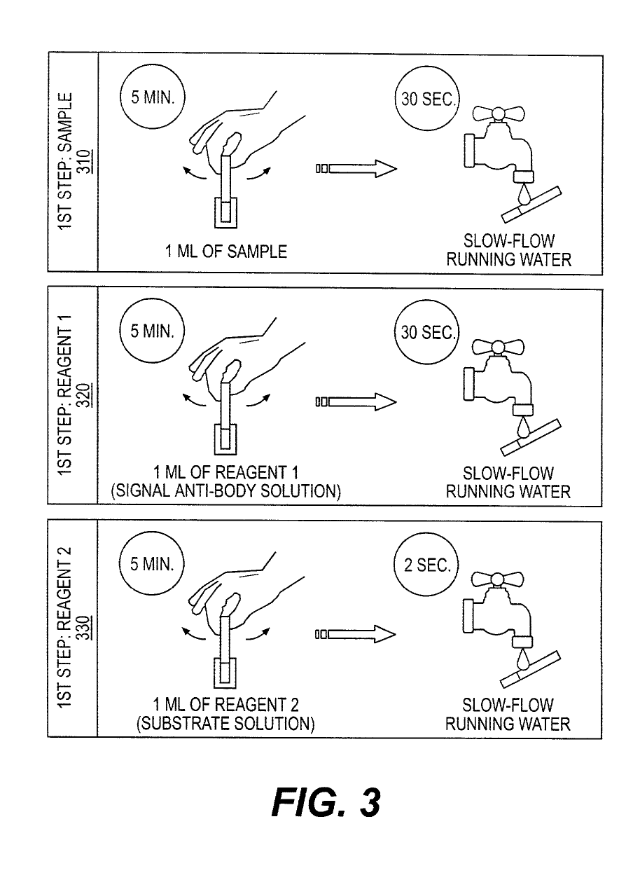

Turning now to FIG. 3, one embodiment of a method of the present invention is illustrated. In a first step 310, insoluble membrane 110 is immersed into a sample, and rinsed. Preferably, insoluble membrane 110 is rinsed with regular tap water for 30 seconds. In a step 320, insoluble membrane 110 is immersed into approximately 1 ml of a first reagent, reagent 1. Reagent 1 is preferably a signal antibody conjugate solution 454 consisting of an antibody 450 conjugated to a horseradish peroxidase enzyme 452. Insoluble membrane 110 is then rinsed with water again, for approximately 20 to 40 seconds. As shown in a step 330, insoluble membrane 110 is immersed into approximately 1 ml of a second reagent, reagent 2. Reagent 2 is preferably a colorless substrate solution consisting of CN-DAB, a stabilized solution of 4-chloro-1-naphthol and 3,3′-diaminobenzidine-4HCL reagent purchased commercially from PIERCE (catalog no. 34000). The CN-DAB colorless substrate solution is prepared by diluting 0.1 ml of CN-DB substrate with 0.9 ml of buffer. Insoluble membrane 110 is rinsed with water for approximately 2 seconds to stop the color reaction, and allowed to air dry completely in a dark place.

FIG. 4 illustrates how an antibody-antigen-antibody complex 460 forms. A peroxidase enzyme 452 is conjugated to an antibody 450 forming the signal antibody 454. A captive antibody 430 is bound to membrane 110 on a test spot 114. A sample protein X 410 links to captive antibody 430 and then to signal antibody 454, thereby forming the antibody-antigen-antibody complex 460. A chemical reaction takes place between peroxidase enzyme 452 and the colorless substrate solution, causing test spot 114 to change color. The chemical reaction causes a colored precipitate to form an antibody-antigen-antibody complex 460, and thus test spot 114 changes color. FIG. 4 also illustrates how control spot 112 changes color after a control anti-peroxidase enzyme antibody 440 links to signal antibody 454 by linking to peroxidase enzyme 452 conjugated to antibody 450, and the chemical reaction between the peroxidase enzyme and the colorless substrate takes place.

Insoluble membrane 110 is inspected to identify any difference in color between the color of test spot 114 and the color of control spot 112. Exemplary test results are shown in FIG. 2. A test spot 114 that shows the same color or more intensity than control spot 112 means that the sample is positive, and contains the protein at a detectable level, as shown in positive test result 210. If test spot 114 shows either less color than control spot 112, or no color at all, then the sample is negative, and does not contain the protein at a detectable level, as shown in negative test result 220. If control spot 112 does not change color or intensity after the test has been completed, then the test is considered invalid and should be repeated carefully. This is because control antibody 440, should react with signal antibody 454 by linking to peroxidase enzyme 452 conjugated to antibody 450 causing a color change at control spot 112. If control spot 112 does not change color, then the test was not conducted properly.

Another aspect of the invention is a kit for detecting the presence of a protein in a sample is provided. The kit has a test strip comprising an insoluble membrane coupled to the test strip, as described above with reference to FIGS. 1 and 2. The kit also includes at least two reagents, the signal antibody solution and the colorless substrate solution, as described above. The insoluble membrane can include a plurality of captive antibodies, wherein these captive antibodies are different from the previously described captive, control and signal antibodies. The plurality of captive antibodies are selected from the group of antibodies consisting of peanut, soy, milk, egg, fish, shellfish, tree nut and seeds. The purpose of having multiple captive antibodies, is that it allows the kit to detect any protein (from those that there is a captive antibody for, from the group described above) that is present in the sample in a detectable level.

The insoluble membrane can further include a plurality of antibodies conjugated to a peroxidase enzyme, wherein the plurality of signal antibody conjugates are different from the previously described signal antibody conjugate, and are different from the previously described captive and control antibodies. The plurality of antibodies conjugated to the peroxidase enzyme are selected from the group of antibodies consisting of peanut, soy, milk, egg, fish, shellfish, tree nut and seeds. Again, The purpose of having multiple signal antibodies, is that it allows the kit to detect any protein (from those that there is a signal antibody for, from the group described above) that is present in the sample in a detectable level.

### METHODS AND EXAMPLES

The following example is provided to explain the invention, and to describe the materials and methods used in carrying out the invention. The example is not intended to limit the invention in any manner.

### Example 1

A test assay was performed using the apparatus and methods of the present invention and a sample containing peanut protein in the range of 0.5 ppm and above. An insoluble membrane was coupled to a support and prepared as follows: a portion of the membrane (the test spot) was coated with purified polyclonal anti-peanut captive antibodies obtained by inoculating rabbits with peanut proteins and then isolating the anti-peanut antibodies from their serum. Another area of the membrane (the control spot) was coated with anti-peroxidase antibodies. After coating the membrane with these antibodies, the membrane was allowed to dry for approximately 30 minutes. The entire membrane was then blocked with a blocking agent of 1% PEG in 5% dry skim milk. The membrane was then rocked for approximately 30 minutes at room temperature. It was washed with PBS and Tween 20 (0.1%) at a pH of 7.4, and rocked for two more minutes. The membrane was washed three times using clean water for each rinse.

After the insoluble membrane was prepared, the entire insoluble membrane was immersed in a sample composed of an extract containing peanut protein in the range of 0.5 ppm and above and then rinsed with running water for approximately 20 to 30 seconds. The insoluble membrane was then contacted with a signal antibody solution composed of an antibody conjugated to a peroxidase enzyme whose ends linked to the anti-peroxidase control antibodies (on the control spot) and to the peanut protein from the sample extract already bound to the captive antibody (on the test spot). The insoluble membrane was then rinsed again with running water for a period of approximately 20 to 30 seconds. The entire insoluble membrane was immersed in a colorless substrate solution, rinsed with running water for a period of approximately 2 seconds and allowed to air dry completely in a dark place. The colorless substrate solution was prepared by diluting 0.1 ml of CN-DB substrate with 0.9 ml of buffer.

After completely drying, the insoluble membrane was inspected to determine if there had been any change in color or intensity of color in the test spot and the control spot. Both the test spot and the control spot changed color after the signal antibody enzyme conjugate bound to the peanut protein already bound to the captive antibody, the signal antibody enzyme conjugate bound to the control antibody, and after the membrane was immersed in the colorless substrate solution. The color and intensity of color of the test spot and control spot were then compared to each other. It was determined that the color of the test spot was more intense than the color of the control spot and therefore it was concluded that the test assay of the present invention successfully detected the presence of peanut protein in the sample at levels as low as 0.5 ppm. After reading and interpreting the test results, the insoluble membrane was placed in a dark place for storage.

### CONCLUSION

While various embodiments of the present invention have been described above, it should be understood that they have been presented by way of example only, and not limitation. Thus, the breadth and scope of the present invention should not be limited by any of the above-described exemplary embodiments, but should be defined only in accordance with the following claims and their equivalents.

What is claimed is:

1. A method for detecting a protein in a sample comprising:
providing an insoluble membrane coupled to a support, wherein the insoluble membrane comprises a captive antibody;
contacting a portion of the insoluble membrane with the sample; and
contacting the insoluble membrane with a signal antibody so that if the protein is present in the sample, the protein links to the captive antibody and to the signal antibody.

2. The method of claim 1, wherein the insoluble membrane further comprises:
a quantity of a control antibody, wherein the control antibody links to the signal antibody.

3. The method of claim 1, further comprising:
contacting the insoluble membrane with a colorless substrate solution.

4. The method of claim 3, wherein the portion changes color after the protein links to the captive antibody and to the signal antibody.

5. The method of claim 2, further comprising:
contacting the insoluble membrane with a colorless substrate solution.

6. The method of claim 5, wherein the quantity changes color after the control antibody links to the signal antibody.

7. The method of claim 5 or 6, wherein the portion and the quantity change color after the control antibody links to the signal antibody.

8. The method of claim 7 further comprising:
comparing the color of the quantity to the color of the portion.

9. The method of claim 1, wherein the protein is selected from the group consisting of peanut, soy, milk, egg, fish, shellfish, tree nuts and seeds.

10. The method of claim 1, wherein the insoluble membrane further comprises:
a second captive antibody, wherein the second captive antibody is different from the captive, signal, and control antibodies.

11. The method of claim 1, wherein the insoluble membrane further comprises:
a plurality of captive antibodies.

12. The method of claim 11, wherein the plurality of captive antibodies are selected from the group of specific antibodies consisting of peanut, soy, milk, egg, fish, shellfish, tree nuts and seeds.

13. The method of claim 1, wherein the insoluble membrane further comprises:
a second signal antibody, wherein the antibody conjugated to the peroxidase enzyme in the second signal antibody is different from the first signal antibody and is different from the captive and control antibodies.

14. The method of claim 1, wherein the insoluble membrane further comprises:
a plurality of signal antibodies.

15. The method of claim 14, wherein the plurality of signal antibodies are selected from the group of antibodies consisting of peanut, soy, milk, egg, fish, shellfish, tree nuts and seeds conjugated to the peroxidase enzyme.

16. The method of claim 1, wherein the step of contacting the portion with the sample is carried out prior to the step of contacting the insoluble membrane with the signal antibody.

17 The method of claim 1, further comprising:
after the step of contacting the portion with sample, rinsing the portion with water.

18. The method of claim 1, further comprising:
after the step of contacting the insoluble membrane with the signal antibody, rinsing the insoluble membrane with water.

19. The method of claim 3, further comprising:
after the step of contacting the insoluble membrane with the colorless substrate solution, rinsing the insoluble membrane with water.

20. The method of claims 17 or 18, wherein the rinsing step is carried out for a period of about 20 to 30 seconds.

21. The method of claim 19, wherein the rinsing step is carried out for about 2 seconds.

22. The method of claim 19, further comprising:
after the step of rinsing the insoluble membrane with water, drying the insoluble membrane.

23. The method of claim 22, wherein the drying step comprises allowing the insoluble membrane to air dry completely in a dark place.

24. The method of claim 2, wherein there is a change to the quantity when the control antibody links to the signal antibody.

25. The method of claim 1, wherein there is a change to the portion when the protein links to the captive antibody and to the signal antibody.

26. The method of claim 22 or 23, further comprising:
after the drying step, inspecting the insoluble membrane to identify any difference between the quantity and the portion.

27. The method of claim 1, wherein the portion changes color when the protein links to the signal antibody.

28. The method of claim 2, wherein the quantity changes color when the control antibody links to the signal antibody.

29. The method of claim 27, wherein the quantity changes color when the control antibody links to the signal antibody.

30. The method of claim 29, further comprising:
comparing the color of the portion to the color of the quantity.

31. A method for detecting a protein in a food processing system comprising:
obtaining a sample of fluid flushed through the food processing system;
providing an insoluble membrane coupled to a support wherein the insoluble membrane comprises a captive antibody;
contacting a portion of the insoluble membrane with the sample; and
contacting the insoluble membrane with a signal antibody so that if the protein is present in the sample, the protein links to the captive antibody and to the signal antibody.

32. The method of claim 31, wherein said protein is a cross-contact protein or a cross-contaminating protein.

33. The method of claim 31, wherein the insoluble membrane further comprises:
a quantity of a control antibody so that the control antibody links to the signal antibody.

34. The method of claim 31, 32, or 33, further comprising:
contacting the insoluble membrane with a colorless substrate solution.

35. The method of claim 31 or 32, wherein the portion changes color after the protein links to the captive antibody and to the signal antibody.

36. The method of claim 33, wherein the quantity changes color after the control antibody links to the signal antibody, and the portion changes color after the protein links to the captive antibody and to the signal antibody.

37. The method of claim 36 further comprising:
comparing the color of the quantity to the color of the portion.

38. The method of claim 31, wherein the insoluble membrane further comprises:
a second captive antibody, wherein the second captive antibody is different from the captive, signal, and control antibodies.

39. The method of claim 31, wherein the insoluble membrane further comprises:
a plurality of captive antibodies.

40. The method of claim 31, 38 or 39, wherein the captive antibody is selected from the group of specific antibodies consisting of peanut, soy, milk, egg, fish, shellfish, tree nuts and seeds.

41. The method of claim 31, wherein the insoluble membrane further comprises:
a second signal antibody, wherein the second signal antibody is different from the captive and signal antibodies.

42. The method of claim 31, wherein the insoluble membrane further comprises:
a plurality of signal antibodies.

43. The method of claim 31, 41 or 42, wherein the signal antibody is selected from the group of antibodies consisting of peanut, soy, milk, egg, fish, shellfish, tree nuts and seeds conjugated to the peroxidase enzyme.

44. The method of claim 31, wherein the protein is selected from the group consisting of peanut, soy, milk, egg, fish, shellfish, tree nuts and seeds.

45. The method of claim 31, wherein the step of contacting the portion with the sample is carried out prior to the step of contacting the insoluble membrane with the signal antibody.

46. The method of claim 31, further comprising:
rinsing the portion with water after the step of contacting the portion with the sample.

47. The method of claim 31, further comprising:
rinsing the insoluble membrane with water after the step of contacting the insoluble membrane with the signal antibody.

48. The method of claim 34, further comprising:
rinsing the insoluble membrane with water after the step of contacting the insoluble membrane with colorless substrate solution.

49. The method of claim 44, further comprising:
after the step of rinsing the insoluble membrane with water, drying the insoluble membrane.

50. The method of claim 49, wherein the drying step comprises:
allowing the insoluble membrane to air dry completely in a dark place.

51. A kit for detecting the presence of a protein in a sample comprising:
a test strip comprising an insoluble membrane coupled to the test strip, wherein the insoluble membrane comprises a captive antibody; and
a reagent comprising a signal antibody, wherein when the test strip is contacted with the sample and with the reagent, if the protein is present in the sample, the protein links to the captive antibody and to the signal antibody.

52. The kit of claim 51, wherein the insoluble membrane further comprises:
a quantity of control antibody, wherein the control antibody links to the signal antibody.

53. The kit of claim 51, wherein the insoluble membrane further comprises:
a second captive antibody, wherein the second captive antibody is different from the captive, signal, and control antibodies.

54. The kit of claim 51, wherein the insoluble membrane further comprises:
a plurality of captive antibodies.

55. The kit of claim 54, wherein the plurality of captive antibodies are selected from the group of specific antibodies consisting of peanut, soy, milk, egg, fish, shellfish, tree nuts and seeds.

56. The kit of claim 51, wherein the insoluble membrane further comprises:
a second signal antibody, wherein the second signal antibody is different from the captive, control, and signal antibodies.

57. The kit of claim 51, wherein the insoluble membrane further comprises:
a plurality of signal antibodies.

58. The kit of claim 57, wherein the plurality of signal antibodies are selected from the group of antibodies consisting of peanut, soy, milk, egg, fish, shellfish, tree nuts and seeds conjugated to the peroxidase enzyme.

59. The kit of claim 51, further comprising:
a second reagent comprising a colorless substrate solution is contacted with the insoluble membrane.

60. The method of claim 4, 25, 27, or 35, wherein the color of the portion will change when the protein, cross-contact protein, or cross-contaminating protein is present in the sample in a level ranging from about 0.5 to about 5 ppm.

61. The method of claim 4, 25, 27, or 35, wherein the color of the portion will change when the protein, cross-contact protein, or cross-contaminating protein is present in the sample in a level ranging from about 5 to about 10 ppm.

62. The method of claim 4, 25, 27, or 35, wherein the color of the portion will change when the protein, cross-contact protein, or cross-contaminating protein is present in the sample in a level greater than about 0.5 ppm.
